(19) United States
(12) Patent Application Publication
(10) Pub. No.: US 2020/0214602 A1
(43) Pub. Date: Jul. 9, 2020

(54) MEASUREMENT APPARATUS

(71) Applicant: Panasonic Intellectual Property Management Co., Ltd., Osaka (JP)

(72) Inventors: KENJI NARUMI, Osaka (JP); SEIJI NISHIWAKI, Hyogo (JP); YASUHIKO ADACHI, Hyogo (JP)

(21) Appl. No.: 16/826,477

(22) Filed: Mar. 23, 2020

Related U.S. Application Data
(63) Continuation of application No. PCT/JP2018/036206, filed on Sep. 28, 2018.

(30) Foreign Application Priority Data
Oct. 25, 2017 (JP) 2017-206083

(51) Int. Cl. A61B 5/1455; A61B 5/145

(52) U.S. Cl. CPC A61B 5/1455; A61B 2562/0238; A61B 5/14546

(57) ABSTRACT

A measurement apparatus includes a light source, an imaging device, a signal processing circuit, and a controller. The controller causes the light source to irradiate a first portion of the scatterer, causes the element to obtain an image signal from a first target area, causes the light source to irradiate a second portion of the scatterer, and causes the element to obtain an image signal from a second target area. The circuit uses the image signals to generate data regarding the position of a target object inside the scatterer. A first distance between the first portion and a center of the first target area is equal to a second distance between the second portion and a center of the second target area, or a difference between the first and second distances is smaller than 10% of a smaller one of the first and second distances.

### BACKGROUND

### 1. Technical Field

The present disclosure relates to a measurement apparatus.

### 2. Description of the Related Art

Techniques have been known in which optical methods are used to diagnose or inspect an internal state of a scatterer and changes thereof, the internal state and the changes being undetectable by the human eye.

For example, Japanese Unexamined Patent Application Publication (Translation of PCT Application) No. 2010-503475 discloses an apparatus that uses an optical method to inspect skin damaged by a burn. Japanese Unexamined Patent Application Publication (Translation of PCT Application) No. 2010-503475 discloses estimating the depth of a burn by using laser speckle analysis.

### SUMMARY

In one general aspect, the techniques disclosed here feature a measurement apparatus for obtaining information about a target object that is present inside a scatterer. The measurement apparatus includes: a light source that emits laser light to the scatterer; an imaging device; a signal processing circuit; and a controller that controls the light source and the imaging device and that controls an irradiation position of the laser light on the scatterer. The controller causes the light source to irradiate a first portion of the scatterer with the laser light and causes the imaging device to output a first image signal representing a first image resulting from the laser light emitted from a first target area that is away from the first portion. The controller causes the light source to irradiate a second portion of the scatterer with the laser light and causes the imaging device to output a second image signal representing a second image resulting from the laser light emitted from a second target area that is away from the second portion. The signal processing circuit generates data regarding a position of the target object inside the scatterer based on the first image signal and the second image signal and outputs the data. A first distance between the first portion and a center of the first target area is equal to a second distance between the second portion and a center of the second target area, or a difference between the first distance and the second distance is smaller than 10% of a smaller one of the first distance and the second distance.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a top view schematically illustrating a configuration example of an analytical model;

FIG. 1B is a side view schematically illustrating the configuration example of the analytical model;

FIG. 2A is one example of a graph obtained by plotting the relationship between the movement distance of a scatterer and a light intensity detected when the scatterer is moved in an X direction;

FIG. 2B is an example of a graph obtained by plotting the relationship between the movement distance of the scatterer and a phase dispersion when the scatterer is moved in the X direction;

FIG. 3A is one example of a graph obtained by plotting the relationship between the movement distance of the scatterer and the change rate of the light intensity relative to values obtained in a surrounding area where a foreign object is not present;

FIG. 3B is an example of a graph obtained by plotting the relationship between the movement distance of the scatterer and the change rate of the phase dispersion relative to values obtained in the surrounding area where a foreign object is not present;

FIG. 4A is an example of a graph obtained by plotting the relationship between the largest value of the change rate of the phase dispersion and the coherence length when the scatterer is scanned;

FIG. 4B is an enlarged graph of a portion up to a coherent length of 150 mm in FIG. 4A;

FIG. 5 is an example of a graph obtained by plotting the relationship between the depth of the foreign object and the largest values of the magnitudes of the change rate of the light intensity and the phase dispersion when the scatterer is scanned;

FIG. 6A is a diagram schematically illustrating a geometric relationship between the measurement apparatus and the scatterer when first image capture in an illustrative embodiment of the present disclosure is performed;

FIG. 6B is a diagram schematically illustrating a geometric relationship between the measurement apparatus and the scatterer when second image capture in the illustrative embodiment of the present disclosure is performed;

FIG. 7A is a view illustrating one example of a first interference image acquired by the first image capture;

FIG. 7B is a view illustrating one example of a second interference image acquired by the second image capture;

FIG. 8 is a flowchart illustrating one example of the operation of the signal processing circuit;

FIG. 9 is a diagram schematically illustrating the configuration of a measurement apparatus in another embodiment of the present disclosure;

FIG. 10A is a top view schematically illustrating a scatterer including foreign objects used in an example;

FIG. 10B is a side view schematically illustrating the scatterer including the foreign objects used in the example;

FIG. 11A is one example of a graph obtained by plotting the relationship between the position of a target area of the scatterer and the light intensity of an interference image;

FIG. 11B is one example of a graph obtained by plotting the relationship between the position of the target area of the scatterer and the phase dispersion of the interference image;

FIG. 12A is one example of a graph obtained by plotting the relationship between the position of the target area of scatterer and the change rate of the light intensity and the change rate of the phase dispersion;

FIG. 12B is one example of a graph obtained by plotting the relationship between the position of the target area of the scatterer and the ratio of the change rate of the light intensity to the change rate of the phase dispersion in the example illustrated in FIG. 12A;

FIG. 12C is one example of a graph obtained by plotting the relationship between the position of the target area of the scatterer and the ratio of he change rates after correction;

FIG. 13A is a graph illustrating a result obtained by extracting, from a result in FIG. 12C, only information about shallow areas where the depth of 2 mm is a reference;

FIG. 13B is a graph illustrating a result obtained by extracting, from the result in FIG. 12C, only information about deep areas where the depth of 5 mm is a reference;

FIG. 14A is a diagram schematically illustrating an example in which a measurement apparatus in the embodiment is applied to detecting blood flow in a head portion of a human body;

FIG. 14B is a view schematically illustrating an example in which a measurement apparatus in the embodiment is applied to detecting subcutaneous blood flow of an arm; and

FIG. 14C is a diagram schematically illustrating an example in which a measurement apparatus in the embodiment is applied to inspecting food products.

### DETAILED DESCRIPTION

### (Findings Underlying Present Disclosure)

Before an embodiment of the present disclosure is described, a description will be given of findings underlying the present disclosure.

Heretofore, various techniques have been developed in which optical methods are used to diagnose or inspect the internal state of a scatterer or changes thereof, the internal state and the changes being undetectable with the human eye. For example, measurement apparatuses, such as photoplethysmographic sensors, near-infrared spectroscopy (NIRS) apparatuses, and optical coherence tomography (OCT) apparatuses, have been developed in addition to the technique disclosed in Japanese Unexamined Patent Application Publication (Translation of PCT Application) No. 2010-503475.

For the photoplethysmographic sensors, the surface of skin is irradiated with light-emitting diode (LED) irradiation light other than green or near-infrared light. Blood including hemoglobin flows in subcutaneous arteries. Hemoglobin has a property of absorbing green or near-infrared light. When the artery pulses, the state of blood flow in the artery changes. As a result, the amount of light absorbed by hemoglobin in an area irradiated with the light changes. When components of irradiation light multiple-scattered by the artery or subcutaneous tissue and emitted from the surface of the skin are detected using a photodiode, it is possible to observe in reception-light intensity changes according to the artery pulsation. Thus, pulse wave signals are detected. The photoplethysmographic sensor detects the interval state of a scatterer as a light intensity.

Another example is a brain function measurement apparatus employing MRS. In NIRS, the scalp is irradiated with near-infrared laser irradiation light or LED irradiation light. Part of irradiation light that is multiple-scattered inside the head portion penetrates the skull and reaches the brain surface layer portion in the skull while scattering. When the amount of hemoglobin contained in the cerebral blood changes, part of the irradiation light is further multiple-scattered in the brain surface layer portion. As a result, the intensity of light emitted from the surface of the scalp changes. This change is observed using a photodiode to thereby estimate the state of activity of the brain surface layer portion. In NIRS, the internal state of a scatterer is also detected as the magnitude of the light intensity, similarly to the photoplethysmographic sensor.

A further example is an optical coherence tomography (OCT) apparatus. The OCT apparatus is one type of optical interferometer. A beam splitter splits irradiation light having a coherence length of about several tens of micrometers into two light beams. One of the light beams is reflected by a mirror to provide reference light. The other light beam is incident on a scatterer, such as a living body, is scattered inside the scatterer, and is emitted from the surface thereof to provide signal light. When the signal light and the reference light are combined together by the beam splitter, interference light is generated through interference of signal light components having an optical path length that is equal to the optical path length of the reference light. The signal light components that contribute to the interference are only light components that travel in straight lines inside the scatterer, are reflected therein, and are emitted from the surface. The light components are referred to as “straight-traveling reflection light”. The signal light components that contribute to the interference do not include light components multiple-scattered inside the scatterer. When the mirror for the reference light is moved in the optical-axis direction to change the optical path length, and the light intensity of the interference light is detected using a photodiode, the distribution of the intensity of reflection light in the depth direction of the scatterer can be measured with a resolution equivalent to the coherence length. The OCT apparatus utilizes an interference phenomenon to detect the intensity of reflection light at a specific depth inside the scatterer.

With a method using the above-described photoplethysmographic sensor, NIRS, or the like, it is difficult to obtain information regarding the depth of a target object that is present inside a scatterer. In addition, although the information regarding the depth of a target object can be obtained with a method using OCT or the like described above, the mirror for the reference light and the beam splitter are required, thus complicating the optical system

The present inventors have found the above-described problems and have conceived a novel measurement apparatus.

The present disclosure includes a measurement apparatus described in the following items.

### [Item 1]

A measurement apparatus according to the present disclosure item 1 is a measurement apparatus for obtaining information about a target object that is present inside a scatterer, and includes:
a light source that emits laser light to the scatterer;
an imaging device;
a signal processing circuit; and
a controller that controls the light source and the imaging device and that controls an irradiation position of the laser light on the scatterer.

The controller
causes the light source to irradiate a first portion of the scatterer with the laser light and causes the imaging device to output a first image signal representing a first image resulting from the laser light emitted from a first target area that is away from the first portion, and
causes the light source to irradiate a second portion of the scatterer with the laser light and causes the imaging device to output a second image signal representing a second image resulting from the laser light emitted from a second target area that is away from the second portion.

The signal processing circuit generates data regarding a position of the target object inside the scatterer based on the first image signal and the second image signal and outputs the data.

A first distance between the first portion and a center of the first target area is equal to a second distance between the second portion and a center of the second target area, or a difference between the first distance and the second distance is smaller than 10% of a smaller one of the first distance and the second distance.

### [Item 2]

In the measurement apparatus according to item b 1,
the controller may include an actuator that changes at least one selected from the group consisting of a position of the light source and a position of the scatterer; and
the controller may drive the actuator to change the irradiation position.

### [Item 3]

In the measurement apparatus according to item 1 or 2,
the signal processing circuit may
   calculate a first average luminance indicating an average of luminance of the first image and a first luminance dispersion indicating a dispersion of luminance of the first image, based on the first image signal,
   calculate a second average luminance indicating an average of luminance of the second image and a second luminance dispersion indicating a dispersion of luminance of the second image, based on the second image signal, and
   generate the data based on the first average luminance, the second average luminance, the first luminance dispersion, and the second luminance dispersion and outputs the data.

### [Item 4]

In the measurement apparatus according to one of item 1 to 3,
the controller may cause the light source to irradiate a third portion of the scatterer with the laser light and cause the imaging device to output a third image signal representing a third image resulting from the laser light emitted from a third target area that is away from the third portion; and
the first distance may be equal to a third distance between the third portion and a center of the third target area, or a difference between the first distance and the third distance may be smaller than 10% of a smaller one of the first distance and the third distance.

### [Item 5]

In the measurement apparatus according to item 4,
the first portion, the second portion, and the third portion may be arranged on a surface of the scatterer in one direction at certain intervals.

### [Item 6]

In the measurement apparatus according to item 1,
the controller may cause the light source to irradiate a third portion of the scatterer with the laser light and cause the imaging device to output a third image signal representing a third image resulting from the laser light emitted from a third target area that is away from the third portion; and
the third portion may be a portion that is different from both the first portion and the second portion and where the target object is not present.

### [Item 7]

The measurement apparatus according to item 7 of the present disclosure is a measurement apparatus for obtaining information about a target object that is present inside a scatterer, and includes:
a light source that emits laser light to the scatterer;
an imaging device;
a signal processing circuit; and
a controller that controls the light source and the imaging device.

The controller causes the light source to irradiate a first portion of the scatterer with the laser light and causes the imaging device to output image signals indicating an image including a first image resulting from the laser light emitted from a first target area that is away from the first portion and a second image resulting from the laser light emitted from a second target area that is away from the first portion.

The signal processing circuit generates data regarding a position of the target object inside the scatterer based on a first signal representing the first image and a second signal representing the second image, the first signal and the second signal being included in the image signals, and outputs the data.

A first distance between the first portion and a center of the first target area is equal to a second distance between the second portion and a center of the second target area, or a difference between the first distance and the second distance is smaller than 10% of a smaller one of the first distance and the second distance.

### [Item 8]

In the measurement apparatus according to item 7,
the signal processing circuit may
   calculate a first average luminance indicating an average of luminance of the first image and a first luminance dispersion indicating a dispersion of luminance of the first image, based on the first signal included in the image signals,
   calculate a second average luminance indicating an average of luminance of the second image and a second luminance dispersion indicating a dispersion of luminance of the second image, based on the second signal included in the image signals, and
   generate the data based on the first average luminance, the second average luminance, the first luminance dispersion, and the second luminance dispersion and outputs the data.

### [Item 9]

In the measurement apparatus according to item 7,
the controller may cause the light source to irradiate a second portion of the scatterer with the laser light and cause the imaging device to output an image signal representing a third image resulting from the laser light emitted from a third target area that is away from the second portion; and
the second portion may be a portion that is different from the first portion and where the target object is not present.

### [Item 10]

In the measurement apparatus according to item 3 or 8,
the signal processing circuit may
   divide an absolute value of a difference between the first average luminance and the second average luminance by the first average luminance or the second average luminance to calculate a change rate of the average luminance,
   divide an absolute value of a difference between the first luminance dispersion and the second luminance dispersion by the first luminance dispersion or the second luminance dispersion to calculate a change rate of the luminance dispersion, and
   determine in which of two or more areas the target object is present, based on a ratio of the change rate of the average luminance to the change rate of the luminance dispersion, the two or more areas being located inside the scatterer and having different depths from a surface of the scatterer.

### [Item 11]

In the measurement apparatus according to one of items 1 to 10,
a coherence length of the laser light may be 1 mm or more and 400 mm or less.

### [Item 12]

In the measurement apparatus according to one of items 1 to 10,
a coherence length of the laser light may be 2 mm or more and 100 mm or less.

### [Item 13]

In the measurement apparatus according to one of items 1 to 10,
a coherence length of the laser light may be 5 mm or more and 20 mm or less.

### [Item 14]

In the measurement apparatus according to one of items 1 to 13,
the scatterer may be a living body; and
the target object may be a portion that is located inside the living body and where a hemoglobin concentration is higher than a hemoglobin concentration in surroundings of the portion.

### [Item 15]

In the measurement apparatus according to one of items 1 to 13,
the scatterer may be food;
the target object may be content contained in the food; and
the signal processing circuit may determine whether or not the content is located at a correct depth inside the food and may output a determination result.

### [Item 16]

In the measurement apparatus according to one of items 1 to 15,
the light source may be capable of switching a coherence length of the laser light between values that are different from each other.

### [Item 17]

In the measurement apparatus according to one of items 1 to 16,
a scattering property of the target object may be different from a scattering property of the scatterer,

In the present disclosure, all or a part of any of circuits, units, apparatuses, devices, parts, or portions or any of functional blocks in the block diagrams may be implemented as one or more of electronic circuits including, but not limited to, a semiconductor device, a semiconductor integrated circuit (IC), or a large-scale integration (LSI). The LSI or IC can be integrated into one chip or also can be a combination of a plurality of chips. For example, functional blocks other than a memory may be integrated into one chip. Although the name used here is an LSI or IC, it may also be called a system LSI, a very large scale integration (VLSI), or an ultra large scale integration (ULSI) depending on the degree of integration. A field programmable gate array (FPGA) that can be programmed after manufacturing an LSI or a reconfigurable logic device that allows reconfiguration of the connection or setup of circuit cells inside the LSI can also be used for the same purpose.

In addition, the functions or operations of all or a part of the circuits, units, apparatuses, devices, parts, or portions can be implemented by executing software. In such a case, the software is recorded on one or more non-transitory recording media, such as a ROM, an optical disk, or a hard disk drive, and when the software is executed by a processor, the software causes the processor together with peripheral devices to execute the functions specified in the software. A system or apparatus may include such one or more non-transitory recording media on which the software is recorded and a processor together with necessary hardware devices such as an interface.

A more specific embodiment in the present disclosure will be described below. However, an overly detailed description may be omitted herein. For example, a detailed description of already well-known things and a redundant description of substantially the same configuration may be omitted herein. This is to avoid the following description becoming overly redundant and to facilitate understanding of those skilled in the art. The accompanying drawings and the following description are provided so as to allow those skilled in the art to fully understand the present disclosure and are not intended to limit the subject matters recited in the claims. In the following description, the same or similar constituent elements are denoted by the same reference numerals.

(Background from which Configuration in Embodiment of Present Disclosure was Derived)

The present inventors studied a measurement apparatus for obtaining information about the depth of a substance that is present inside an optical scatterer having a scattering property that is different from that of the substance. Study was conducted based on an analytical model of such a measurement apparatus. The substance is hereinafter referred to as a “foreign object” or a “target object”. By using a Monte Carlo method, the present inventors calculated the distribution of multiple scattering light emitted from the surface of a scatterer in the analytical model when the scatterer is irradiated with irradiation light.

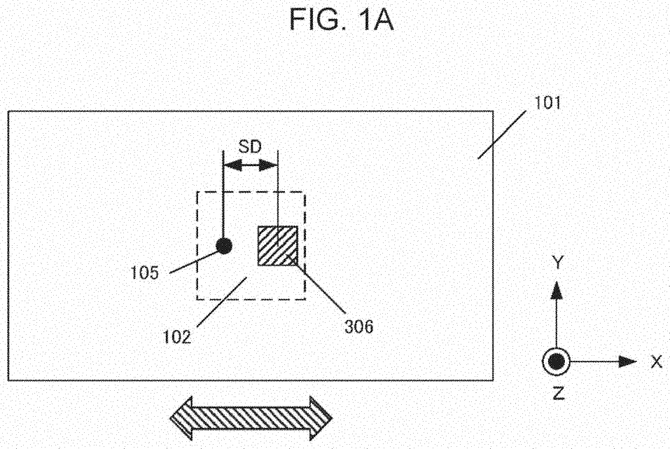

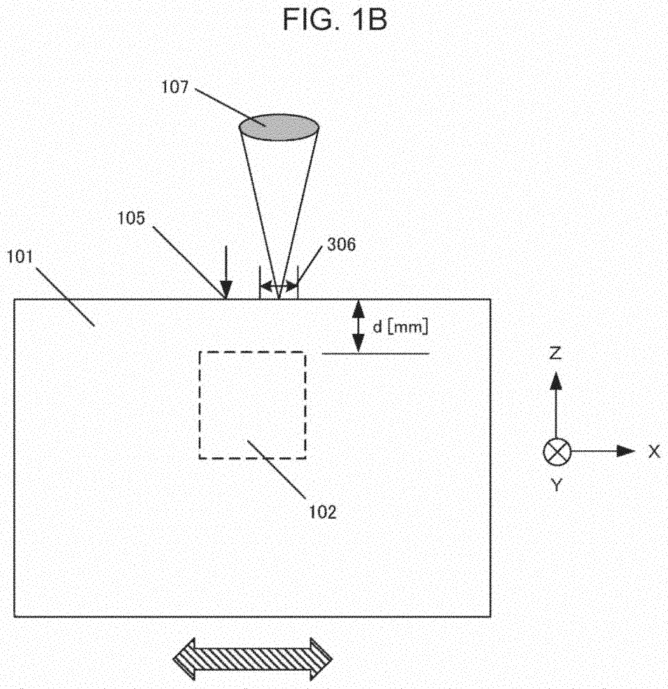

FIG. 1A and FIG. 1B are a top view and a side view, respectively, schematically illustrating the configuration of the analytical model. The hatched double-headed arrows illustrated in FIGS. 1A and 1B represent directions in which an optical scatterer 101 is moved. A coordinate system defined by X-, Y-, and Z-axes that are orthogonal to each other is used in the following description. In this analytical model, a surface of the scatterer 101 is parallel to the XY plane. A portion (hereinafter referred to as an “irradiation portion 105”) on the surface of the scatterer 101 is irradiated with irradiation light, and light that is emitted from a portion (hereinafter referred to as a “target area 306”) that is a predetermined-distance away from the irradiation portion 105 in the X direction is detected.

In this analytical model, a foreign object 102, which is a measurement target, is contained at a position at a depth d [mm] inside the scatterer 101. The scatterer 101 is a material having an absorption coefficient of μₐ=0.002 mm⁻¹ and a reduced scattering coefficient of μₛ=0.24 mm⁻¹. The size of the foreign object 102 is 10×10×10 mm. The foreign object 102 is a substance having a low-scattering property than the scatterer 101 and has a reduced scattering coefficient μₛ of zero. Light with a sufficiently small-size spot is used as the irradiation light.

An area that is 4 mm away from the irradiation portion 105 in the X direction and that has a size of 3.2×3.2 mm is selected as an area to be photographed, that is, the target area 306. The distance between the irradiation portion 105 and the center of the target area 306 is hereinafter referred to as a “source-detector distance”. In this analytical model, the source-detector distance is 4 mm. Scatter light emitted from the target area 306 forms an image through an optical system 107 having a numerical aperture (NA) of 1.0. The depth d from the surface of the scatterer 101 to the surface of the foreign object 102 was changed to three depths: 2 mm, 5 mm, and 10 mm. Light intensities and optical-phase spatial dispersions (hereinafter referred to as “phase dispersions”) in an image plane (not illustrated) corresponding to the target area 306 were calculated for the respective depths. In this analysis, standard deviations of the light intensities in the image plane was calculated as the phase dispersions.

Light that enters the inside of a scatterer is multiple-scattered, and the optical path length of each light ray varies on a far greater order of magnitude than the wavelength of the irradiation light. Thus, the phase in the image plane becomes spatially random. However, there is a possibility that the random phase dispersions can be observed as interference images. The possibility changes depending on the relationship between the coherence length of the irradiation light and the average optical path length of a large number of light rays that pass through the scatterer.

The absolute quantity of variations in the optical path lengths of a large number of light rays that pass through the scatterer 101 (the absolute quantity is hereinafter referred to as “optical-path-length deviation”) increases, as the average optical path length increases. When the coherence length of the irradiation light is greater than or equivalent to the optical-path-length deviation, two light rays that are adjacent to each other in the image plane interfere with each other. Thus, the phase difference between the two light rays appears in an interference image. On the other hand, when the coherence length of the irradiation light is smaller than the optical-path-length deviation, the probability that the two light rays that are adjacent to each other in the image plane interfere with each other decreases. Thus, the probability that the phase difference appears in an interference image also decreases.

Hence, the average optical path length of light rays that are multiple-scattered from when the light rays from the irradiation portion 105 enter the inside of the scatterer 101 until the light rays reach the target area 306 is reflected in the phase dispersion observed as an interference image.

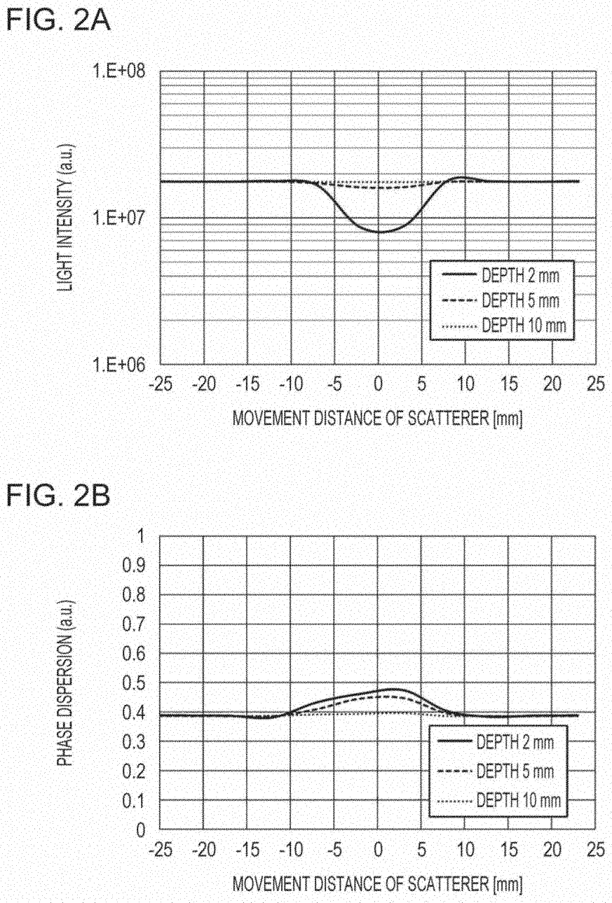

FIG. 2A is one example of a graph obtained by plotting the relationship between the movement distance of the scatterer 101 and a light intensity detected when the scatterer 101 is moved in the X direction. The example illustrated in FIG. 2A shows that when an image of an area inside which the foreign object 102 is present is captured, the light intensity that is detected decreases, compared with a case in which an image of another area is captured. This can be described as follows. The foreign object 102 has a low-scattering property than the scatterer 101. This reduces the amount of light that returns from the portion where the foreign object 102 is present toward the surface of the scatterer 101.

FIG. 2B is an example of a graph obtained by plotting the relationship between the movement distance of the scatterer and the phase dispersion when the scatterer 101 is moved in the X direction. The example illustrated in FIG. 2B shows that when an image of an area inside which the foreign object 102 is present is captured, the phase dispersion increases, compared with a case in which an image of another area is captured. This can be described as follows. Owing to the influence of reductions in the optical path lengths of light that passes through a portion where the foreign object 102 is present, the average optical path length of light rays that pass through the inside of the scatterer decreases from the irradiation portion to the target area. As a result, the optical-path-length deviation decreases relative to the coherence length of the irradiation light, thereby increasing the interference.

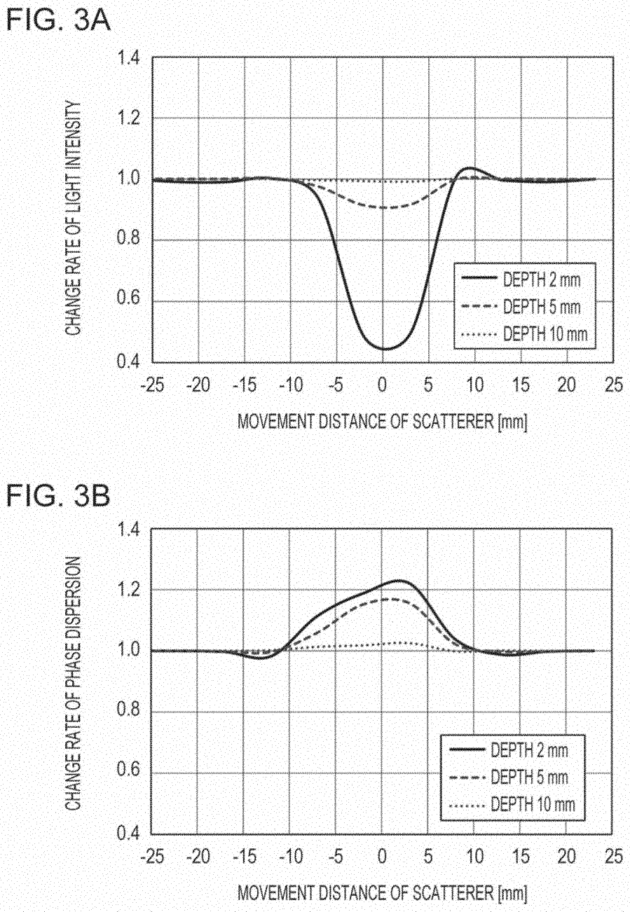

FIG. 3A is an example of a graph obtained by plotting the relationship between the movement distance of the scatterer 101 and the change rate of the light intensity relative to values obtained in a surrounding area where the foreign object 102 is not present. FIG. 3B is an example of a graph obtained by plotting the relationship between the movement distance of the scatterer 101 and the change rate of the phase dispersion relative to values obtained in the surrounding area where the foreign object 102 is not present.

The examples illustrated in FIGS. 3A and 3B show that the light intensity and the phase dispersion both exhibit a larger change rate for a smaller depth of the foreign object 102. What is noteworthy is that the change rate of the light intensity decreases sharply with respect to the depth, whereas the change rate of the phase dispersion decreases gradually with respect to the depth.

In this analysis, the coherence length of irradiation light is 10 mm. When the coherence length is too small, the interference of the multiple scattering light decreases. Thus, changes in the phase dispersion depending on the presence/absence of a foreign object decrease. On the other hand, when the coherence length is too large, the interference of the multiple scattering light stays strong regardless of the presence/absence of a foreign object. Thus, changes in the phase dispersion decrease. Therefore, an appropriate coherence length is in a certain range.

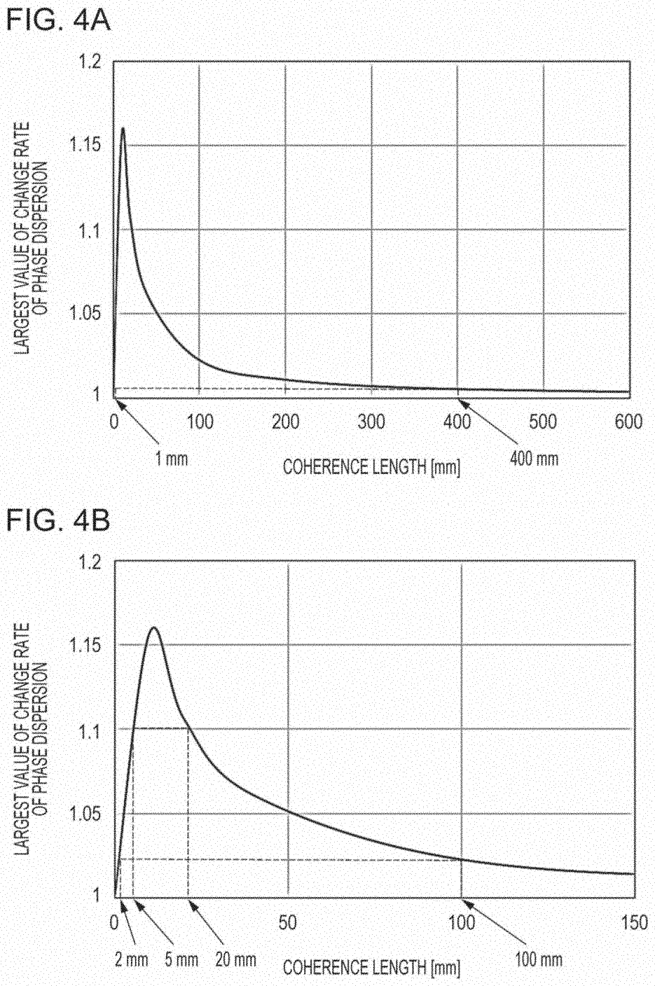

FIG. 4A is an example of a graph obtained by plotting the relationship between the largest value of the change rate of the phase dispersion and the coherence length when the scatterer is scanned. FIG. 4B is an enlarged graph of a portion up to a coherent length of 150 mm in FIG. 4A.

The example illustrated in FIG. 4A and FIG. 4B shows that the range of the coherence length with which the change rate of the phase dispersion increases is, for example, 1 mm or more and 400 mm or less, 2 mm or more and 100 mm or less in one example, and is 5 mm or more and 20 mm or less in another example.

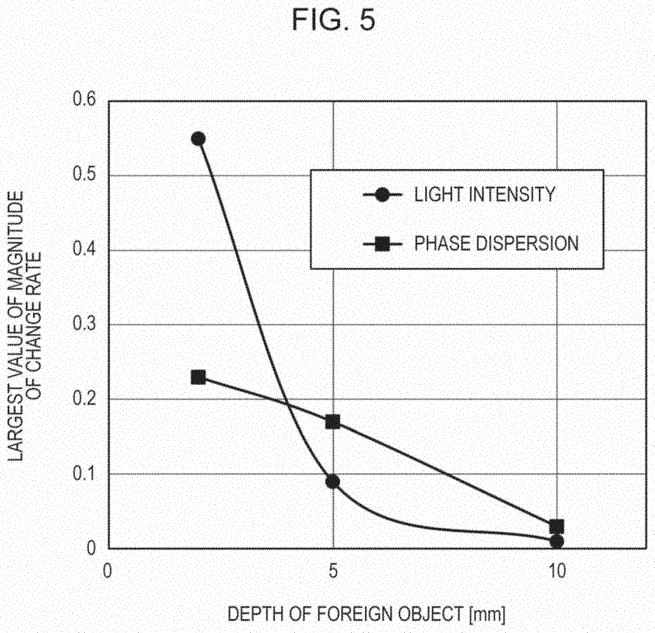

FIG. 5 is an example of a graph obtained by plotting the relationship between the depth of the foreign object 102 and the largest values of the magnitudes of the change rate of the light intensity and the phase dispersion when the scatterer 101 is scanned, In this case, the magnitudes of the change rates are the absolute values of change rates relative to respective references. Each circle mark indicates the largest value of the magnitude of the change rate of the light intensity, and each square mark indicates the largest value of the magnitude of the change rate of the phase dispersion. In other words, the graph in FIG. 5 illustrates how much sensitivity the detections based on the light intensity and the phase dispersion have with respect to the depth of the foreign object 102.

The example illustrated in FIG. 5 shows that, in this analytical model, when the depth of the foreign object is 2 mm, the detection based on the light intensity has a higher sensitivity, and when the depth of the foreign object is 5 mm or 10 mm, the detection based on the phase dispersion has a higher sensitivity.

The reason why the decrease tendency with respect to the depth of the foreign object 102 varies between the change rate of the light intensity and the change rate of the phase dispersion is thought to be as follows. When the irradiation light is incident on the surface of the scatterer 101, the number of light rays that reach a deeper portion from the surface decreases. The change rate of the light intensity due to the foreign object 102 that is present at a certain depth directly reflects the number of light rays that reach the depth.

On the other hand, the change rate of the phase dispersion reflects changes in the average optical path length from the irradiation portion 105 to the target area 306, When the foreign object 102 is present at a very shallow portion, the optical path lengths of light rays that are scattered inside the foreign object 102 decrease. However, since the foreign object 102 has a low-scattering property, the number of light rays that are scattered inside the foreign object 102 and that return to the surface also decreases. As a result, the effect of the decrease in the average optical path length is thought to be limited.

It was found that when the foreign object 102 having a scattering property that is different from that of the scatterer 101 is present inside the scatterer 101, the change rate of the light intensity and the change rate of the phase dispersion exhibit different decrease tendencies with respect to the depth of the foreign object 102.

In this analytical model, the foreign object 102 has a lower-scattering property than the scatterer 101. However, the configuration with which the phenomenon in which the change tendency with respect to the depth differs between the change rate of the light intensity and the change rate of the phase dispersion occurs is not limited to the above-described configuration. For example, when the foreign object 102 has a higher-scattering property than the scatterer 101, the light intensity can increase and the phase dispersion can decrease in an area where the foreign object 102 is present inside the scatterer 101. In any configuration with which at least one of the absorption coefficient and the reduced scattering coefficient differs between the scatterer 101 and the foreign object 102, the phenomenon in which the change tendency with respect to the depth differs can occur.

### (Configuration and Operation in Embodiment)

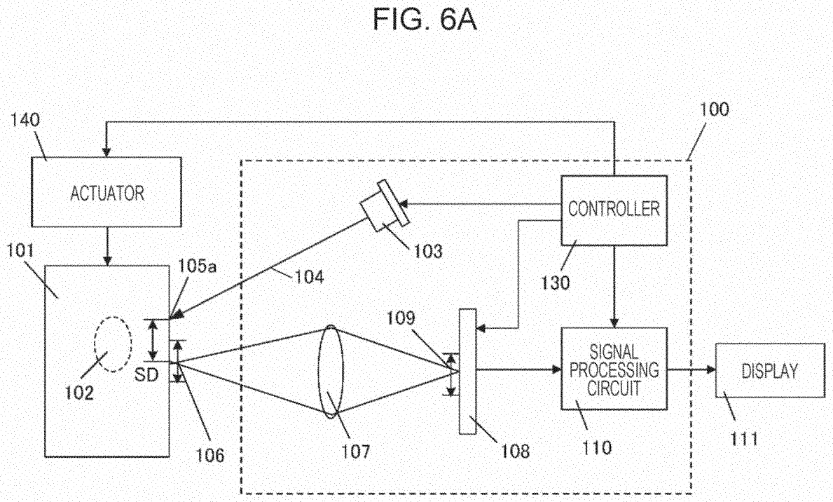

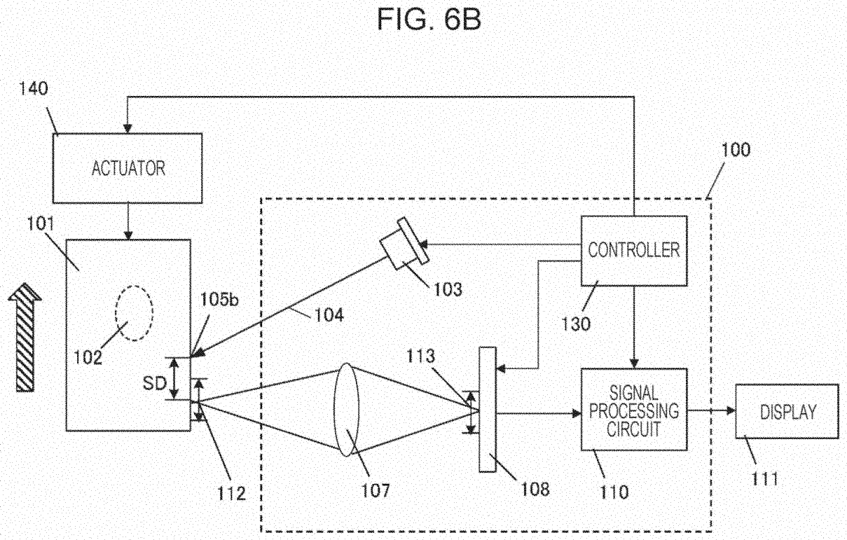

FIG. 6A is a diagram schematically illustrating the configuration of a measurement apparatus 100 in the illustrative embodiment of the present disclosure. FIG. 6A also schematically depicts an optical scatterer 101. The measurement apparatus 100 performs first image capture while irradiating a first portion on the surface of the scatterer 101 with light and then performs second image capture while irradiating a second portion on the surface of the scatterer 101 with the light. FIG. 6A schematically illustrates a geometric relationship between the measurement apparatus 100 and the scatterer 101 when the first image capture is performed. FIG. 6B schematically illustrates a geometric relationship between the measurement apparatus 100 and the scatterer 101 when the second image capture is performed. The hatched upward arrow in FIG. 6B represents a direction in which the scatterer 101 is moved. As illustrated in FIGS. 6A and 6B, the target area for the first image capture and the target area for the second image capture differ from each other.

The measurement apparatus 100 in the present embodiment includes a light source 103, an imaging device 108, a signal processing circuit 110, and a controller 130.

The light source 103 emits light 104 having a coherence length of 1 mm or more and 400 mm and less to the scatterer 101. As described above, the light source 103 may emit light having, for example, a coherence length of 2 mm or more and 100 mm or less. In one example, the light source 103 may emit light having a coherence length of 5 mm or more and 20 mm or less. The light source 103 may be, for example, a laser light source that emits laser light having a coherence length in any of the ranges described above.

The controller 130 includes a control circuit that controls the light source 103 and the imaging device 108. The control circuit in the controller 130 controls an actuator 140 to thereby control a light irradiation position of the scatterer 101, The actuator 140 is coupled to a table on which the scatterer 101 is placed and enables the scatterer 101 to move in a direction parallel to an image capture plane of the imaging device 108.

The controller 130 causes the light source 103 to irradiate a first portion 105a of the scatterer 101 with the light 104. In this state, the controller 130 causes the imaging device 108 to output a first image signal representing a first interference image 109, which is a first image resulting from light emitted from a first target area 106 that is a distance SD away from the first portion 105a. Upon the output of the first image signal, the first image capture is completed,

Subsequently, the controller 130 causes the light source 103 to irradiate a second portion 105b of the scatterer 101 with the light 104. In this state, the controller 130 causes the imaging device 108 to output a second image signal representing a second interference image 113, which is a second image resulting from light emitted from a second target area 112 that is away from the second portion 105b. Upon the output of the second image signal, the second image capture is completed.

The signal processing circuit 110 performs computational operation using the first image signal and the second image signal to generate data regarding the depth of a foreign object 102 that is present inside the scatterer 101 and outputs the data.

The controller 130 executes the above-described control, for example, by executing a program recorded in a memory. The controller 130 may include, for example, an integrated circuit, such as a central processing unit (CPU) or a microcomputer. The controller 130 and the signal processing circuit 110 may be integrated into a single unit.

In FIGS. 6A and 6B, the actuator 140 is illustrated as a constituent element that is independent from the controller 130. However, the controller 130 may include the actuator 140. In response to a control signal given from the control circuit in the controller 130, the actuator 140 causes the position of the scatterer 101 to move in a direction that intersects an optical axis of an optical system 107. In the present embodiment, the controller 130 drives the actuator 140 to thereby change the irradiation position, as described above.

In the present embodiment, the distance between the first portion 105a and the center of the first target area 106 is equal to the distance between the second portion 105b and the center of the second target area 112, Thus, when the first portion 105a, the first target area 106, the second portion 105b, and the second target area 112 are present in an area where the foreign object 102 is not present, the detection light intensity in the first image capture and the detection light intensity in the second image capture can be made to have approximately the same value. Herein, two distances being “equal” means that the absolute value of the difference between the two distances is smaller than 10% of the smaller one of the two distances. The distance between the first portion 105a and the center of the first target area 106 may be different from the distance between the second portion 105b and the center of the second target area 112. In such a case, the signal processing circuit 110 may be adapted to correct at least one of the first and second image signals in accordance with the difference between the distances, so as to allow comparison of the first and second image signals.

In the example illustrated in FIG. 6A, the foreign object 102 is present inside the scatterer 101. The foreign object 102 is a substance in which at least one of the absorption coefficient and the reduced scattering coefficient is different from the corresponding coefficient(s) of the scatterer 101 around the foreign object 102. One example of the scatterer 101 may be physiological tissue, and one example of the foreign object 102 may be hemoglobin in blood.

The operation of the measurement apparatus 100 in the present embodiment will be described below.

In accordance with an instruction from the controller 130, the light source 103 irradiates the first portion 105a on the surface of the scatterer 101 with the light 104, which has a predetermined coherence length, Scatter light emitted from the first target area 106 that is a distance SD away from the first portion 105a passes through the optical system 107, and an image of the scatter light is formed in the image capture plane of the imaging device 108. The imaging device 108 captures the formed image as a first interference image 109 and outputs a first image signal representing the interference image 109.

Next, as illustrated in FIG. 6B, the control circuit in the controller 130 causes the actuator 140 to move the scatterer 101 while maintaining the positions of the light source 103, the optical system 107, which includes a lens, and the imaging device 108. The imaging device 108 then captures an image of interference light from the second target area 112, which is different from the first target area 106. The light from the light source 103 irradiates the second portion 105b, which is different from the first portion 105a. The image of the interference light from the second target area 112, which is the distance SD away from the second portion 105b, is formed in the image capture plane of the imaging device 108. The imaging device 108 captures the image as a second interference image 113 and outputs a second image signal representing the second interference image 113.

In the present embodiment, the foreign object 102 is present directly inside the first target area 106 of the scatterer 101, and the foreign object 102 is absent directly inside the second target area 112. Thus, signal processing using the first image signal and the second image signal can provide information about the depth at which the foreign object 102 is present. It is sufficient as long as the foreign object 102 is present directly inside one of the first target area 106 and the second target area 112.

There are also cases in which, in actual image capture, at which position inside the scatterer 101 the foreign object 102 is present is unknown. In such a case, the first target area 106 and the second target area 112 may be selected based on images of a large number of target areas, the images being captured while moving the scatterer 101. In such a case, the controller 130 causes the light source 103 to sequentially irradiate a plurality of portions including the first portion 105a and the second portion 105b of the scatterer 101 with light. The controller 130 causes the imaging device 108 to sequentially output image signals indicating interference images resulting from light emitted from target areas that are located at an equal distance from the respective portions, The signal processing circuit 110 performs computational operation using the image signals to thereby generate data regarding the depth of a target object and outputs the data. Those portions are typically arranged at regular intervals in one direction along the surface of the scatterer 101, but does not necessarily have to be arranged at regular intervals.

The signal processing circuit 110 calculates a light intensity and a phase dispersion with respect to each of the first interference image 109 and the second interference image 113 by using a method described below, and estimates at what degree of depth the foreign object 102 is present, based on the calculation result, The signal processing circuit 110 outputs information regarding the depth onto a display 111. Details of signal processing performed by the signal processing circuit 110 are described later.

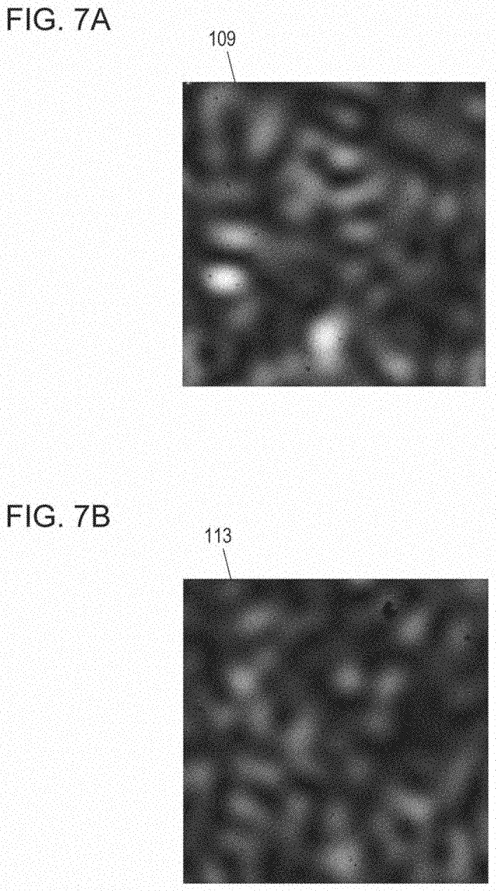

FIG. 7A is a view illustrating one example of the first interference image 109. FIG. 7B is a view illustrating one example of the second interference image 113. As illustrated in FIGS. 7A and 7B, the first interference image 109 and the second interference image 113 are observed as speckle images. Light rays that are adjacent to each other form a random-luminance distribution in the image plane of each speckle image. The distribution of random luminance reflects the distribution of random phase differences. Hence, the speckle images can be regarded as reflecting the distribution of random phase differences, An optical element that can detect phase differences may be disposed on the imaging device 108 to directly detect the distribution of the phase differences. In such a case, the detected distribution of the phase differences may be used as an interference image.

Next, the operation of the signal processing circuit 110 will be described in more detail.

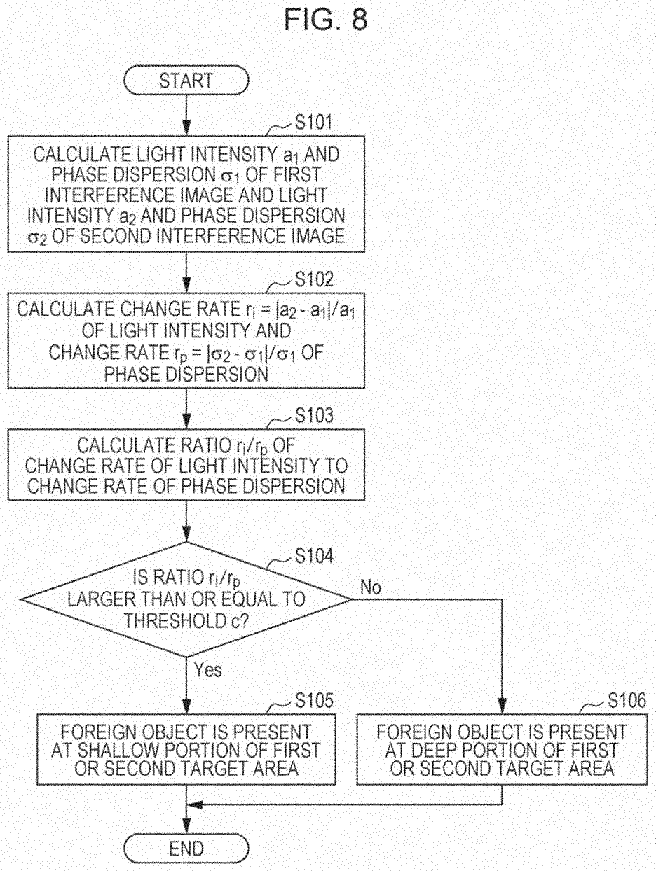

FIG. 8 is a flowchart illustrating one example of the operation of the signal processing circuit 110.

In step S101, the signal processing circuit 110 calculates a light intensity a₁ and a phase dispersion σ₁ of the first interference image 109 and a light intensity a₂ and a phase dispersion σ₂ of the second interference image 113.

The light intensity represents an average luminance of the interference image and is also referred to as an “average luminance”. The average luminance can be determined by, for example, calculating an average value of luminance values of the entire area of the interference image.

The phase dispersion represents a luminance dispersion of the interference image and is also referred to as a “luminance dispersion”. The luminance dispersion can be determined by, for example, a method described below.

(1) The area of the interference image is divided into a plurality of areas according to a two-dimensional grid pattern, and average values of luminances are determined for the respective areas.
(2) Using all of the divided areas as a parameter, a standard deviation of the average values of the luminances is determined as a phase dispersion.

A distribution may be used instead of the standard deviation. Another value that numerically expresses the degree of dispersion in the luminance distribution may also be used as the phase dispersion.

In step S102, the signal processing circuit 110 calculates a change rate of the light intensity and a change rate of phase dispersions. The change rate of the light intensity can be determined by computational operation rᵢ=|a₂−a₁|/a₁. The change rate of the phase dispersion can be determined by computational operation rₚ=|σ₂−σ₁|/σ₁.

In step S103, the signal processing circuit 110 calculates a ratio rᵢ/rₚ of the change rate of the light intensity to the change rate of the phase dispersion.

In step S104, the signal processing circuit 110 determines whether or not the ratio rᵢ/rₚ is larger than or equal to a predetermined threshold c. The threshold c is appropriately determined according to image-capture conditions, such as optical properties of the scatterer 101 and the foreign object 102, the coherence length of the light source 103, and the source-detector distance.

When the ratio rᵢ/rₚ is larger than or equal to the threshold c, the signal processing circuit 110 determines that the foreign object 102 is present at a relatively shallow portion of the first target area 106 or the second target area 112 (step S105). On the other hand, when the ratio rᵢ/rₚ is smaller than the threshold c, the signal processing circuit 110 determines that the foreign object 102 is present at a relatively deep portion of the first target area 106 or the second target area 112 (step S106). The inside of the scatterer 101 may be divided into areas at three or more different depths, and in which depth area the foreign object 102 is present may be determined based on the value of the ratio rᵢ/rₚ.

The signal processing circuit 110 sends data indicating a result of the determination made in step S105 or S106 to the display 111. The signal processing circuit 110 may send the data to a recording medium (not illustrated) instead of the display 111. The display 111 displays the result of the determination. For example, the display 111 displays at least one of an image of the relatively deep portion in the scatterer 101 and an image of the shallow portion in the scatterer 101. In still another example, at least one of the probability that the foreign object 102 is present at the relatively deep portion in the scatterer 101 and the probability that the foreign object 102 is present at the relatively shallow portion in the scatterer 101 is displayed as a numerical value. In a further example, the scatterer 101 is a living body, and at least one of information regarding blood flow at the relatively shallow portion and information regarding blood flow at the relatively deep portion is displayed as a numerical value. In yet another example, at least one of an optical property at the relatively deep portion in the scatterer 101 and an optical property of the relatively shallow portion in the scatterer 101 is displayed as a numerical value. The optical property is, for example, the intensity of reflection light. In yet another example, the information displayed as the numerical value is displayed, for instance, as “normal” or “abnormal or an index converted into symbols, such as “○”, “Δ”, or “x”. In a yet further example, the above-described image(s) or information may be sent to an analyzing device, inspection equipment, a computer, or the like that is located external to the measurement apparatus 100, instead of being displayed.

As described above, the signal processing circuit 110 in the present embodiment executes operations described below.

(1) A first average luminance indicating the average of luminance of the first interference image 109 and a first luminance dispersion indicating a luminance dispersion of the first interference image 109 are determined based on the first image signal.
(2) A second average luminance indicating the average of luminance of the second interference image 113 and a second luminance dispersion indicating a luminance dispersion of the second interference image 113 are calculated based on the second image signal.
(3) Computational operation using the first and second average luminances and the first and second luminance dispersions is performed to generate data regarding the depth of the target object, and the data is output. The “data regarding the depth” is not limited to the above-described example and may be any data or signal indicating the degree of the depth of a measurement target object from the surface of the scatterer.

More specifically, in step (3) described above, the signal processing circuit 110 executes operations described below.

(3a) The absolute value of the difference between the first average luminance and the second average luminance is divided by the first or second average luminance to thereby calculate a change rate of the average luminance.
(3b) The absolute value of the difference between the first luminance dispersion and the second luminance dispersion is divided by the first or second luminance dispersion to thereby calculate a change rate of the luminance dispersion.
(3c) In which area of two or more different depth areas inside the scatterer 101 the foreign object 102 is present is determined based on the ratio of the change rate of the average luminance to the change rate of the luminance dispersion.

With the above-described configuration and operations in the present embodiment, information regarding the depth of a target object that is present inside a scatterer can be obtained with a simple optical system.

In the present embodiment, the actuator 140, which moves the scatterer 101, is used in order to change the position of the first target area 106 and the position of the second target area 112. An actuator that moves the light source 103, the optical system 107, and the imaging device 108 may be used, instead of moving the scatterer 101. It is sufficient as long as the actuator has a mechanism that changes the position of the scatterer 101 relative to the light source 103 and the imaging device 108 in the measurement apparatus 100. The actuator may be included in the controller 130 or may be an element external to the controller 130.

Also, a configuration described below may be employed instead of the configuration for moving the scatterer 101.

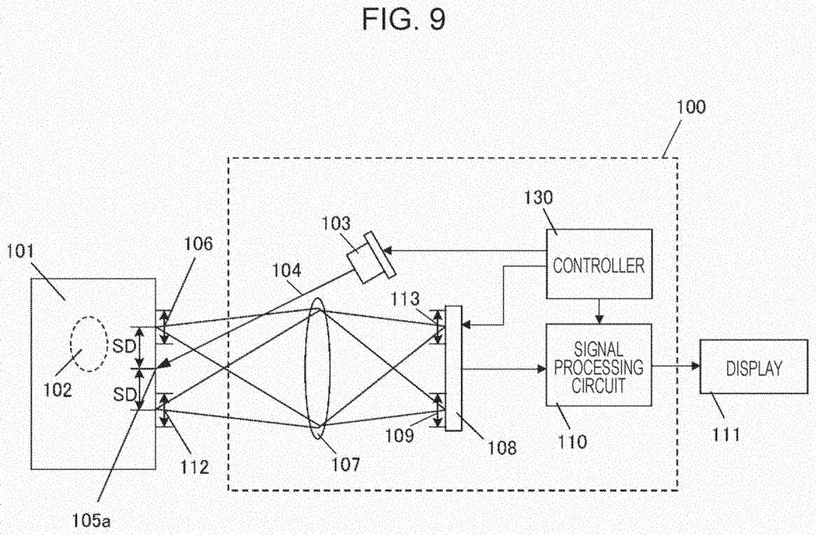

FIG. 9 is a diagram schematically illustrating the configuration of a measurement apparatus 100 in another embodiment of the present disclosure. The measurement apparatus 100 acquires images of a plurality of target areas at a time without light scanning.

Differences from the embodiment described above are mainly described below.

The measurement apparatus 100 in the present embodiment does not include an actuator. A controller 130 causes a light source 103 to irradiate a first portion 105a of a scatterer 101 with light 104. The controller 130 causes an imaging device 108 to output image signals indicating an image including a first interference image 109 resulting from light emitted from a first target area 106 that is away from the first portion 105a and a second interference image 113 resulting from light emitted from a second target area 112 that is away from the first portion 105a. As a result, two rounds of image capture are completed.

A signal processing circuit 110 performs computational operation using a signal representing the first interference image 109 and a signal representing the second interference image 113, the signals being included in the image signals, to generate data regarding the depth of a foreign object 102, which is a target object that is present inside the scatterer 101, and outputs the data.

The distance between the first portion 105a and the center of the first target area 106 is equal to the distance between the first portion 105a and the center of the second target area 112. The distance between the first portion 105a and the center of the first target area 106 may be different from the distance between the first portion 105a and the center of the second target area 112. In such a case, in accordance with the difference between the distances, the signal processing circuit 110 may be adapted to be able to correct at least one of the signal representing the first interference image 109 and the signal representing the second interference image 113, the signals being included in the image signal, to make it possible to compare both the signals.

The signal processing circuit 110 in the present embodiment executes operations described below.

(1) A first average luminance indicating an average luminance of the first interference image 109 and a first luminance dispersion indicating a luminance dispersion of the first interference image 109 are calculated based on the signal representing the first interference image 109.
(2) A second average luminance indicating an average luminance of the second interference image 113 and a second luminance dispersion indicating a luminance dispersion of the second interference image 113 are calculated based on the signal representing the second interference image 113.
(3) Computational operation using the first and second average luminances and the first and second luminance dispersions are performed to generate data regarding the depth of the foreign object 102 that is present inside the scatterer 101, and the data is output. A specific method for generating the data is the same as or similar to that described above.

As illustrated in FIG. 9, a target area whose image can be captured by the optical system 107 and the imaging device 108 includes both the first target area 106 and the second target area 112. Both the first target area 106 and the second target area 112 are a distance SD away from the first portion 105a. The imaging device 108 may crop an area corresponding to the first interference image 109 and an area corresponding to the second interference image 113 from an entire area whose image can be captured. With this configuration, it is possible to omit the process for moving the scatterer 101 in order to capture images of different target areas. Accordingly, it is possible to more easily obtain information regarding a depth inside a scatterer.

### EXAMPLE

A description will be given of an example in which calculation was performed in order to confirm the advantages of the present disclosure.

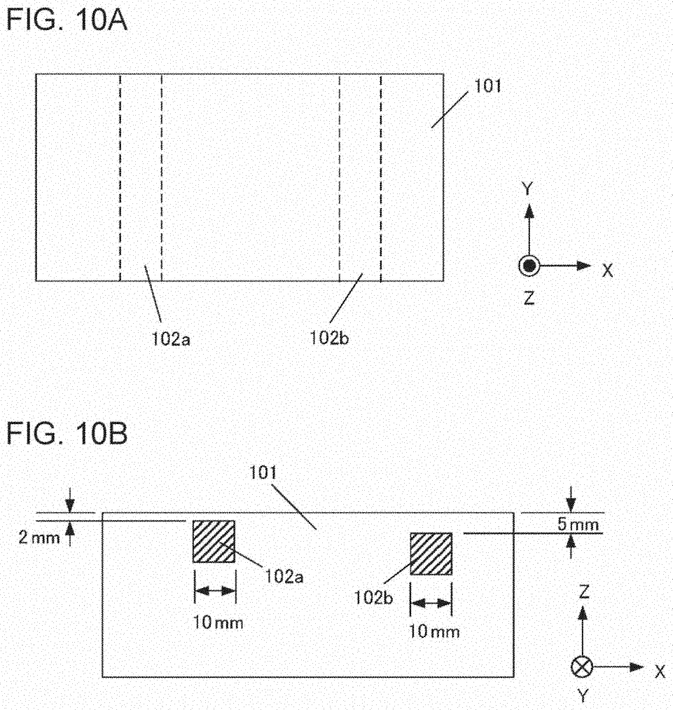

FIGS. 10A and 10B are a top view and a side view, respectively, schematically illustrating a scatterer 101 including foreign objects 102a and 102b used in the example.

In this example, two 10-millimeter-square bar-shaped foreign objects 102a and 102b are embedded in the scatterer 101. The absorption coefficient and the reduced scattering coefficient of the scatterer 101 are the same as the absorption coefficient and the reduced scattering coefficient of the scatterer 101 described above in the example illustrated in FIGS. 1A and 1B. Similarly, the absorption coefficient and the reduced scattering coefficient of each of the foreign objects 102a and 102b are the same as the absorption coefficient and the reduced scattering coefficient of the foreign object 102 described above in the example illustrated in FIGS. 1A and 1B. The depth of the foreign object 102a from the surface of the scatterer 101 is 2 mm, and the depth of the foreign object 102b from the surface of the scatterer 101 is 5 mm.

With the exemplary configuration of the measurement apparatus 100 in the above-described embodiment, the scatterer 101 is irradiated with irradiation light, the scatterer 101 is moved 25 steps at intervals of 5 mm in the X direction, and images of a total of 25 target areas are captured. A method that is similar to the method in the above-described embodiment was used to calculate a light intensity and a phase dispersion on the basis of the luminance distributions of the interference images corresponding to the respective target areas.

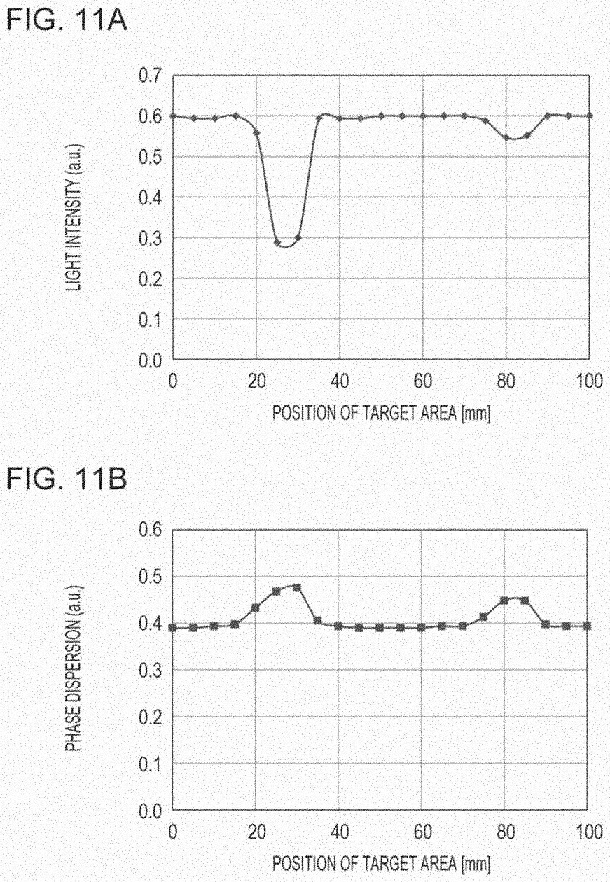

FIG. 11A is one example of a graph obtained by plotting the relationship between the position of the target area of the scatterer and the light intensity of the interference image. FIG. 11B is one example of a graph obtained by plotting the relationship between the position of the target area of the scatterer and the phase dispersion of the interference image. Each of the graphs displays changes due to the presence of the foreign object 102a at a depth of 2 mm and the foreign object 102b at a depth of 5 mm inside the scatterer.

In the following description, the position of the foreign object 102a at a depth of 2 mm and the position of the foreign object 102b at a depth of 5 mm are separated based on the light intensity and the phase dispersion.

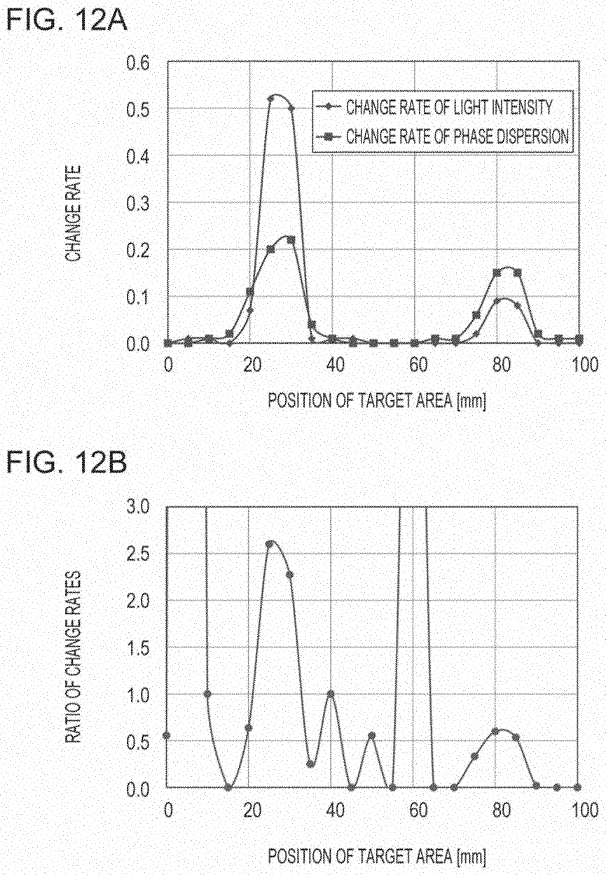

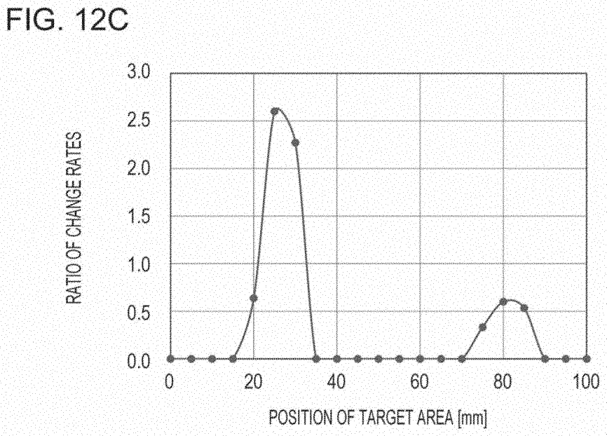

FIG. 12A is one example of a graph obtained by plotting the relationship between the position of a target area of a scatterer and the change rate of the light intensity and the change rate of the phase dispersion. The change rate of the light intensity and the change rate of the phase dispersion correspond to rᵢ and rₚ, respectively, in the embodiment described above. The light intensity and the phase dispersion at the position of 0 mm on the scatterer are respectively used as references for the change rate of the light intensity and the change rate of the phase dispersion, In this case, the position for the references does not necessarily have to be 0 mm and may be a position under which no foreign object is present. For example, the position for the references may be a position (such as an end of the scatterer 101) where the foreign object 102 is presumed to be absent. Also, the references may or may not be designated on the scatterer 101, or another scatterer that has the same optical property and that does not include the foreign object 102 may be used for the references.

FIG. 12B is one example of a graph obtained by plotting the relationship between the position of the target area of the scatterer and the ratio of the change rate of the light intensity to the change rate of the phase dispersion in the example illustrated in FIG. 12A, The ratio corresponds to rᵢ/rₚ in the embodiment, As illustrated in FIG. 12B, variations are considerably large with respect to the position of the scatterer. This is because when the change rate of the phase dispersion, the change rate being the denominator of rᵢ/rₚ, approaches zero, the value of rᵢ/rₚ drastically increases, regardless of the value of the change rate of the light intensity.

When the value of the change rate of the phase dispersion is close to zero, a foreign object is assumed to be absent or is assumed to be present at a depth where it is not detectable. Accordingly, it is rational to estimate that no foreign object is present at the depth of 2 mm or 5 mm. In the example, for rᵢ/rₚ<0.05, correction for setting the ratio of the change rates to zero was performed.

FIG. 12C is one example of a graph obtained by plotting the relationship between the position of a target area of the scatterer and the ratio of the change rates after the correction. As illustrated in FIG. 12C, a rational result was obtained.

Lastly, a description will be given of graph separation based on whether or not the corrected ratio of the change rates is larger than a certain value.

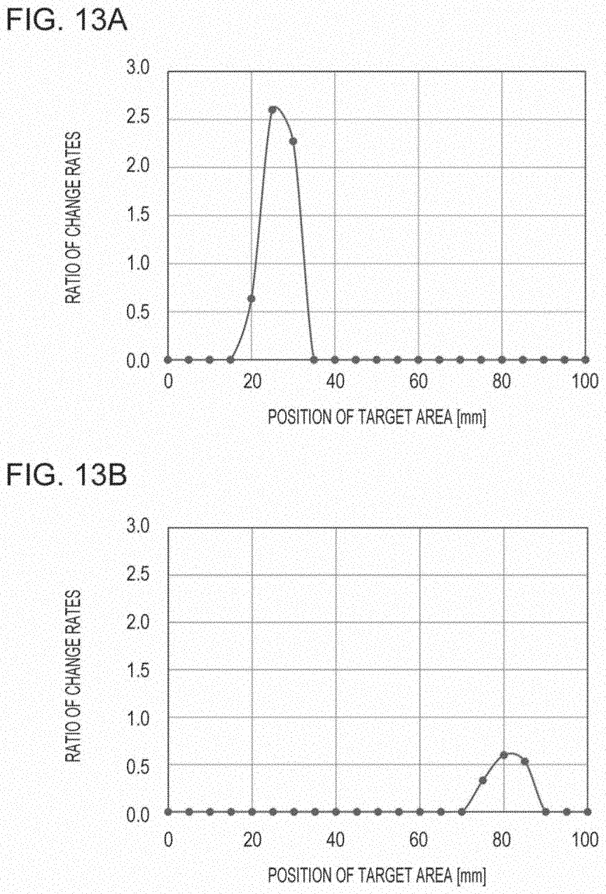

FIG. 13A is a graph illustrating a result obtained by extracting, from the result in FIG. 12C, only information about shallow areas where the depth of 2 mm is a reference. FIG. 13B is a graph illustrating a result obtained by extracting, from the result in FIG. 12C, only information about deep areas where the depth of 5 mm is a reference. In the example, when the corrected ratio of the change rates is larger than 0.6, the ratio of the change rates is reflected in an image for a depth of 2 mm, and when the corrected ratio of the change rates is smaller than or equal to 0.6, the ratio of the change rates is reflected in an image for a depth of 5 mm. Thus, one-dimensional image of a foreign object can be separated by using the depth as a reference.

In the above-described embodiment, the respective change rates are calculated based on the light intensity and the phase dispersion, and information about the depth is obtained based on the ratio of the change rate of the light intensity to the change rate of the phase dispersion. However, the information may be obtained based on a difference or a magnitude relationship, not the ratio. The information regarding the depth may also be obtained based on the light intensity and the phase dispersion, instead of the change rate of the light intensity and the change rate of the phase dispersion. Also, another calculation method may be used in accordance with the optical properties of the scatterer or the foreign object(s). In addition, the relationship between the state of a foreign object and the change rate of the light intensity and the change rate of the phase dispersion may be machine-learned, and the information regarding the depth may be obtained based on the result of the learning. Also, the relationship between the state of a foreign object and the light intensity and the phase dispersion may be machine-learned, and the information regarding the depth may be obtained based on the result of the learning.

In an actual scatterer, the absorption properties and the scattering properties of foreign objects that are present inside the scatterer may be different from each other. In such a case, a method in which images of a target area are captured by irradiating the scatterer while changing the coherence length of a light source to a plurality of lengths is effective in order to obtain the information regarding the depths of respective foreign objects. The reason will be described below.

For example, two foreign objects, that is, a foreign object having a low-scattering property and a foreign object having a high-scattering property, are assumed to be present inside a scatterer. The average optical path length of light that travels through the foreign object having a high-scattering property becomes larger than the average optical path length of light that travels through the foreign object having a low-scattering property. This means that the optical-path-length deviation of light that travels through the foreign object having a high-scattering property increases. In order to cause interference to occur in the image plane with respect to the light, a light source having a larger coherence length is necessary. In other words, it can be said that the relationship between the coherence length of a light source and the interference changes depending on the scattering property of a foreign object.

Hence, it can be said that the use of a light source with different coherence lengths makes it possible to obtain the information regarding the depth even when foreign objects having different diffusion properties are present inside a scatterer. Also, the same concept can be applied to a case in which foreign objects having absorption properties that are different from each other are present inside a scatterer.

An actual scatterer may also have an absorption property and a scattering property that are not spatially uniform. In such a case, an average light intensity and a spatial distribution of the phase dispersion are pre-measured in a target area where there is apparently no foreign object. Next, the average light intensity and the phase dispersion in a target area where there is a foreign object are measured. It is possible to obtain the information regarding the depth by finding positions that are respectively spatially close to a target area where no foreign object is present and a target area where a foreign object is present and calculating the change rates of average light intensities and the change rates of phase dispersions with respect to the positions.

The configurations of the measurement apparatus used in the embodiment and the example are not limited to the above-described configurations, The configuration of the measurement apparatus may be changed to an appropriate configuration within a scope in which the disclosed configuration and the advantages are satisfied.

### (Application Example)

An application example of the measurement apparatus according to the present embodiment will be described below.

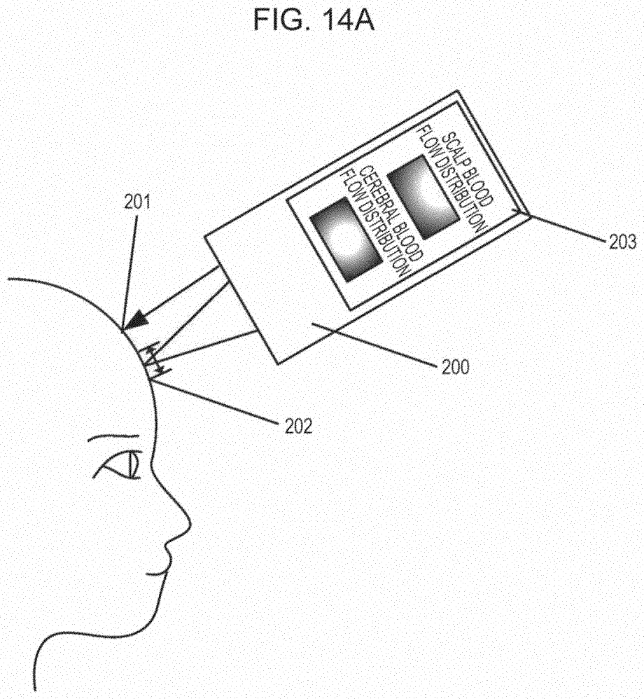

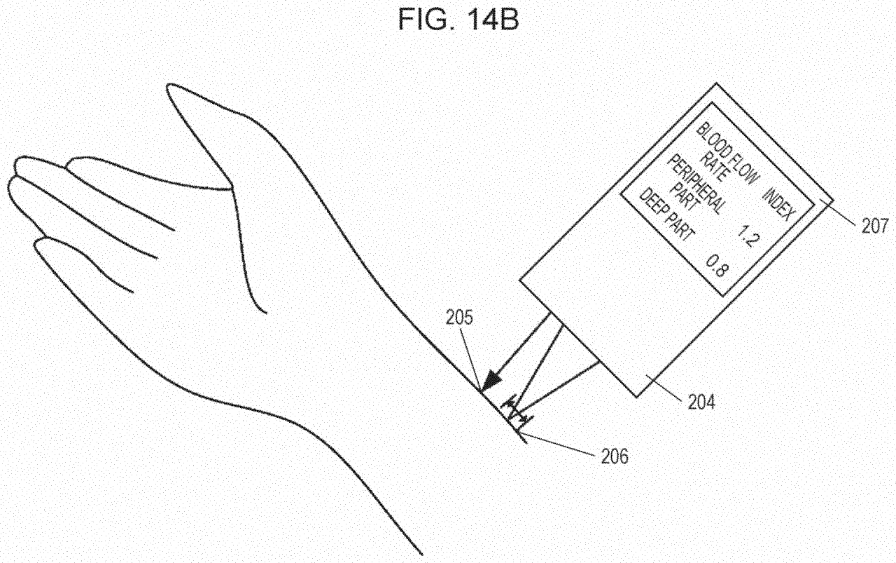

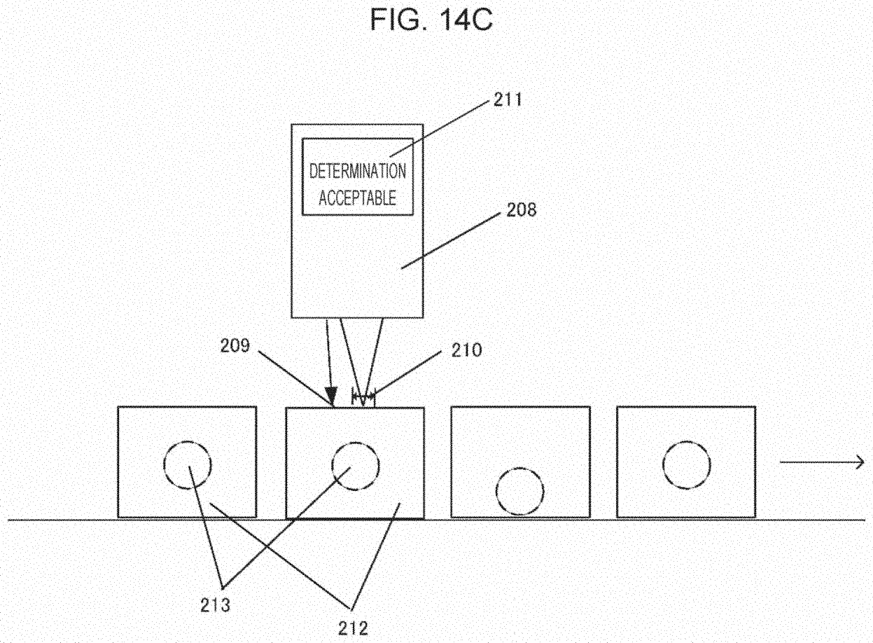

FIG. 14A is a diagram schematically illustrating an example in which a measurement apparatus 200 in the embodiment is applied to detecting blood flow in a head portion 201 of a human body. As illustrated in FIG. 14A, the head portion 201 is irradiated with irradiation light of the measurement apparatus 200, and an interference image of multiple scattering light from an area 202 located on the surface of the scalp and corresponding to the first or second target area.

In the example illustrated in FIG. 14A, the head portion 201 corresponds to the scatterer. Optical absorption due to hemoglobin is high at a portion where the hemoglobin concentration is higher than that in its surroundings owing to blood flow, and the intensity of reflection light from the portion is small. Thus, that portion is thought to correspond to the target object. A signal processing circuit (not illustrated) in the measurement apparatus 200 generates data indicating the state of blood in the scatterer and outputs the data.

Use of the measurement apparatus 200 in the embodiment makes it possible to separately detect the distribution of blood flow at a deep portion from the surface of the scalp and the distribution of blood flow in a brain surface layer portion located at a shallow portion from the surface of the scalp. A display 203 may display those blood flow distributions.

FIG. 14B is a view schematically illustrating an example in which a measurement apparatus 204 in the embodiment is applied to detecting subcutaneous blood flow of an arm 205. As in the case of the head portion, the arm 205 or the wrist is irradiated with irradiation light from the measurement apparatus 204, and an interference image of multiple scattering light is captured from an area 206 located on the skin surface and corresponding to the first or second target area.

In the example in FIG. 14B, the arm 205 or the wrist corresponds to the scatterer, As in the case of the head portion, a portion where the hemoglobin concentration due to blood flow is higher than that in its surroundings is thought to correspond to a target object. A signal processing circuit (not illustrated) in the measurement apparatus 204 generates data indicating the state of blood in the scatterer and outputs the data.

Use of the measurement apparatus 204 in the embodiment makes it possible to separately detect the distribution of blood flow in a peripheral blood vessel part located at a shallow portion from the surface of skin and the distribution of blood flow in an artery part or vein part located at a deep portion. A display 207 may display the blood flow distribution as a numerical value corresponding to a blood flow rate.

Temporally continuously obtaining the distribution of the hemoglobin concentration and performing computational operation makes it possible to estimate a pulse waveform, a blood pressure value, and so on of only a deep artery part or only a shallow peripheral blood vessel part.

FIG. 14C is a diagram schematically illustrating an example in which a measurement apparatus 208 in the embodiment is applied to inspecting food products 212.

In the example in FIG. 14C, each food product 212 corresponds to the scatterer. In an inspection process after content 213 is contained in each food product 212, the food product 212 in which the content 213 is placed at its center is regarded as an acceptable product. When the absorption coefficient of the content 213 differs from the absorption coefficient of the food product 212, the intensity of reflection light from the content 213 differs from the intensity of reflection light from the food product 212. Accordingly, the content 213 is thought to be a target object. A surface 209 of each food product 212 is irradiated with irradiation light from the measurement apparatus 208, and an interference image of multiple scattering light from an area 210 corresponding to the first or second target area is captured. A signal processing circuit (not illustrated) in the measurement apparatus 208 determines whether or not the content 213 is positioned at a correct depth and outputs a determination result.

With the measurement apparatus 208 in the embodiment, whether or not the content 213 is placed at a correct depth can be determined based on the information regarding the depth of the content 213 viewed from the image capture side, and a determination can be made as to whether each food product 212 is an acceptable product or unacceptable product. A display 211 may display the determination result.

What is claimed is:

1. A measurement apparatus for obtaining information about a target object that is present inside a scatterer, the measurement apparatus comprising:
a light source that emits laser light to the scatterer;
an imaging device;
a signal processing circuit; and
a controller that controls the light source and the imaging device and that controls an irradiation position of the laser light on the scatterer, wherein
the controller causes the light source to irradiate a first portion of the scatterer with the laser light and causes the imaging device to output a first image signal representing a first image resulting from the laser light emitted from a first target area that is away from the first portion;
the controller causes the light source to irradiate a second portion of the scatterer with the laser light and causes the imaging device to output a second image signal representing a second image resulting from the laser light emitted from a second target area that is away from the second portion;
the signal processing circuit generates data regarding a position of the target object inside the scatterer based on the first image signal and the second image signal and outputs the data; and
a first distance between the first portion and a center of the first target area is equal to a second distance between the second portion and a center of the second target area, or a difference between the first distance and the second distance is smaller than 10% of a smaller one of the first distance and the second distance.

2. The measurement apparatus according to claim 1, wherein
the controller includes an actuator that changes at least one selected from the group consisting of a position of the light source and a position of the scatterer; and
the controller drives the actuator to change the irradiation position.

3. The measurement apparatus according to claim 1,
wherein the signal processing circuit
calculates a first average luminance indicating an average of luminance of the first image and a first luminance dispersion indicating a dispersion of luminance of the first image, based on the first image signal,
calculates a second average luminance indicating an average of luminance of the second image and a second luminance dispersion indicating a dispersion of luminance of the second image, based on the second image signal, and
generates the data based on the first average luminance, the second average luminance, the first luminance dispersion, and the second luminance dispersion and outputs the data.

4. The measurement apparatus according to claim 1, wherein
the controller causes the light source to irradiate a third portion of the scatterer with the laser light and causes the imaging device to output a third image signal representing a third image resulting from the laser light emitted from a third target area that is away from the third portion; and
the first distance is equal to a third distance between the third portion and a center of the third target area, or a difference between the first distance and the third distance is smaller than 10% of a smaller one of the first distance and the third distance.

5. The measurement apparatus according to claim 4,
wherein the first portion, the second portion, and the third portion are arranged on a surface of the scatterer in one direction at certain intervals.

6. The measurement apparatus according to claim 1, wherein
the controller causes the light source to irradiate a third portion of the scatterer with the laser light and causes the imaging device to output a third image signal representing a third image resulting from the laser light emitted from a third target area that is away from the third portion; and
the third portion is a portion that is different from both the first portion and the second portion and where the target object is not present.

7. A measurement apparatus for obtaining information about a target object that is present inside a scatterer, the measurement apparatus comprising:
a light source that emits laser light to the scatterer;
an imaging device;
a signal processing circuit; and
a controller that controls the light source and the imaging device, wherein
the controller causes the light source to irradiate a first portion of the scatterer with the laser light and causes the imaging device to output image signals indicating an image including a first image resulting from the laser light emitted from a first target area that is away from the first portion and a second image resulting from the laser light emitted from a second target area that is away from the first portion;
the signal processing circuit generates data regarding a position of the target object inside the scatterer based on a first signal representing the first image and a second signal representing the second image, the first signal and the second signal being included in the image signals, and outputs the data; and
a first distance between the first portion and a center of the first target area is equal to a second distance between the second portion and a center of the second target area, or a difference between the first distance and the second distance is smaller than 10% of a smaller one of the first distance and the second distance.

8. The measurement apparatus according to claim 7,
wherein the signal processing circuit
calculates a first average luminance indicating an average of luminance of the first image and a first luminance dispersion indicating a dispersion of luminance of the first image, based on the first signal,
calculates a second average luminance indicating an average of luminance of the second image and a second luminance dispersion indicating a dispersion of luminance of the second image, based on the second signal, and
generates the data based on the first average luminance, the second average luminance, the first luminance dispersion, and the second luminance dispersion and outputs the data.

9. The measurement apparatus according to claim 7, wherein
the controller causes the light source to irradiate a second portion of the scatterer with the laser light and causes the imaging device to output an image signal representing a third image resulting from the laser light emitted from a third target area that is away from the second portion; and
the second portion is a portion that s different from the first portion and where the target object is not present.

10. The measurement apparatus according to claim 3,
wherein the signal processing circuit
divides an absolute value of a difference between the first average luminance and the second average luminance by the first average luminance or the second average luminance to calculate a change rate of the average luminance,
divides an absolute value of a difference between the first luminance dispersion and the second luminance dispersion by the first luminance dispersion or the second luminance dispersion to calculate a change rate of the luminance dispersion, and
determines in which of two or more areas the target object is present, based on a ratio of the change rate of the average luminance to the change rate of the luminance dispersion, the two or more areas being located inside the scatterer and having different depths from a surface of the scatterer.

11. The measurement apparatus according to claim 1,
wherein a coherence length of the laser light is 1 mm or more and 400 mm or less.

12. The measurement apparatus according to claim 1,
wherein a coherence length of the laser light is 2 mm or more and 100 mm or less.

13. The measurement apparatus according to claim 1,
wherein a coherence length of the laser light is 5 mm or more and 20 mm or less.

14. The measurement apparatus according to claim 1,
wherein the scatterer is a living body; and
the target object is a portion that is located inside the living body and where a hemoglobin concentration is higher than a hemoglobin concentration in surroundings of the portion.

15. The measurement apparatus according to claim 1,
wherein the scatterer is food;
the target object is content contained in the food; and
the signal processing circuit determines whether or not the content is located at a correct depth inside the food and outputs a determination result.

16. The measurement apparatus according to claim 1,
wherein the light source is capable of switching a coherence length of the laser light between values that are different from each other.

17. The measurement apparatus according to claim 1,
wherein a scattering property of the target object is different from a scattering property of the scatterer.
